(19) United States
(12) Patent Application Publication
(10) Pub. No.: US 2026/0000523 A1
(43) Pub. Date: Jan. 1, 2026

(54) AN IMPLANT INSERTION SYSTEM

(71) Applicant: SIGNATURE ORTHOPAEDICS EUROPE LTD, Dublin (IE)

(72) Inventors: Declan BRAZIL, Lane Cove West (AU); William DUONG, Dublin (IE); Andrew SHIMMIN, Dublin (IE); Michael O'SULLIVAN, Dublin (IE); Jonathan BARE, Dublin (IE); Dermot COLLOPY, Dublin (IE)

(21) Appl. No.: 18/880,709

(22) PCT Filed: Jul. 27, 2023
(86) PCT No.: PCT/AU2023/050687

§ 371 Date: Jan. 2, 2025

(30) Foreign Application Priority Data
Jul. 28, 2022 (AU) 2022902117

(51) Int. Cl. A61F 2/46; A61F 2/34

(52) U.S. Cl. CPC A61F 2/4609; A61F 2/34; A61F 2002/3401; A61F 2002/3493; A61F 2002/4627; A61F 2310/00179

(57) ABSTRACT

An implant system has an acetabular cup with an interior bearing surface with a central bearing zone that is smooth and devoid of holes or indentations. The cup also has a peripheral bearing zone that contains insertion tool engagement holes. The engagement holes are only present in the peripheral zone and have specific shapes and dimensions. The system includes an insertion tool with retractable interlocks that fit within the engagement holes to position the cup during implantation. The central bearing zone bears the main forces during articulation, while the peripheral zone withstands minor forces. The location of the engagement holes has a negligible effect on the cup's performance. The cup can be made of ceramic and may have a coated exterior surface for osseointegration.

### FIELD OF THE INVENTION

This invention relates generally to apparatus and techniques for placing and positioning acetabular cups.

### BACKGROUND OF THE INVENTION

An acetabular cup is inserted into a prepared acetabulum to accept an articulating component therein, either a femoral head component directly or via a liner for dual-mobility hip prosthesis.

The acetabular cup is normally driven into the acetabulum using an insertion tool comprising a rounded head at a distal end of a rod to avoid damage to the smooth bearing surface of the acetabular cup.

However, it is difficult to correctly position the acetabular cup once in the acetabulum. For example, using the aforedescribed insertion tool, the acetabular cup cannot be rotated axially or tilted, potentially resulting in malpositioning.

Whereas metallic cups may be provided with intricate tool engagement holes such as screw holes within the rim thereof to engage corresponding screws of custom engagement tools, it is difficult to make such tool engagement holes in ceramic acetabular cups without damaging the structural integrity thereof and/or damaging the articular surface thereof which may be highly polished.

As such, one type of conventional acetabular cup engagement tool involves a plate which seals across the rim of the acetabular cup and which is coupled to a vacuum to generate suction force to hold the acetabular cup to the engagement tool. However, the suction force is generally insufficient, especially for the removal of the acetabular cup and cannot be used to twist the acetabular cup.

The present invention seeks to provide a way to overcome or substantially ameliorate at least some of the deficiencies of the prior art, or to at least provide an alternative.

It is to be understood that, if any prior art information is referred to herein, such reference does not constitute an admission that the information forms part of the common general knowledge in the art, in Australia or any other country.

### SUMMARY OF THE DISCLOSURE

There is provided herein an implant system comprising an acetabular cup defining an interior bearing surface. The interior bearing surface defines a central bearing zone that is entirely smooth and a peripheral bearing zone surrounding the central bearing zone.

The interior bearing surface has a plurality of insertion tool engagement holes located only in the peripheral bearing zone.

The system involves the use of an insertion tool comprising a rod and a plurality of radially operative interlocks at a distal end of the rod.

The interlocks are configurable in a non-engaging configuration wherein the interlocks are retracted in towards the rod to fit within the peripheral bearing zone and an engaging configuration wherein the interlocks extend out from the rod to engage respective insertion tool engagement holes to attach the acetabular cup to the insertion tool.

The central bearing zone, preferably located within 150° of the centroid of articulation, bears the main forces during articulation. It is smooth and free from holes or indentations to withstand these predominant forces. On the other hand, the peripheral bearing zone only experiences minor or infrequent forces at extreme rotations.

The engagement holes are located only in the peripheral bearing zone, which has a negligible effect on the performance of the interior bearing surface.

As such, the present system allows for insertion and manipulation of the acetabular cup using the insertion tool without significantly compromising the performance of the bearing surface once the cup is implanted.

Furthermore, the protrusions avoid contacting the central bearing zone, thereby avoiding damage to the smooth surface thereof, which may be highly polished.

The present system is especially suited for ceramic acetabular cups wherein the engagement holes can be formed in ceramic without substantially affecting the structural integrity of the acetabular cup.

Other aspects of the invention are also disclosed.

### BRIEF DESCRIPTION OF THE DRAWINGS

Notwithstanding any other forms which may fall within the scope of the present invention, preferred embodiments of the disclosure will now be described, by way of example only, with reference to the accompanying drawings in which:

FIG. 1 shows a cross-sectional view of an acetabular cup through a centroid thereof in accordance with an embodiment;

FIG. 2 shows a perspective view of the acetabular cup;

FIG. 3 shows a top cross-sectional view of the acetabular cup;

FIG. 4 shows a further cross-sectional view of the acetabular cup;

FIG. 5 shows a magnified view of a tool engagement hole of the acetabular cup in accordance with an embodiment;

FIG. 6 shows an acetabular cup comprising tool engagement holes in accordance with a first embodiment;

FIG. 7 shows an acetabular cup comprising tool engagement holes in accordance with a second embodiment;

FIG. 8 shows an acetabular cup comprising tool engagement holes in accordance with a preferred embodiment;

FIG. 9 shows an acetabular cup comprising a unitary tool engagement hole in accordance with a third embodiment

FIG. 10 shows an implant system involving an insertion tool which engages the acetabular cup;

FIG. 11 shows a magnified view illustrating how a protrusion of the insertion tool engages a respective engagement hole of the acetabular cup; and

FIG. 12 shows a cross-sectional view of the system of FIG. 10.

### DESCRIPTION OF EMBODIMENTS

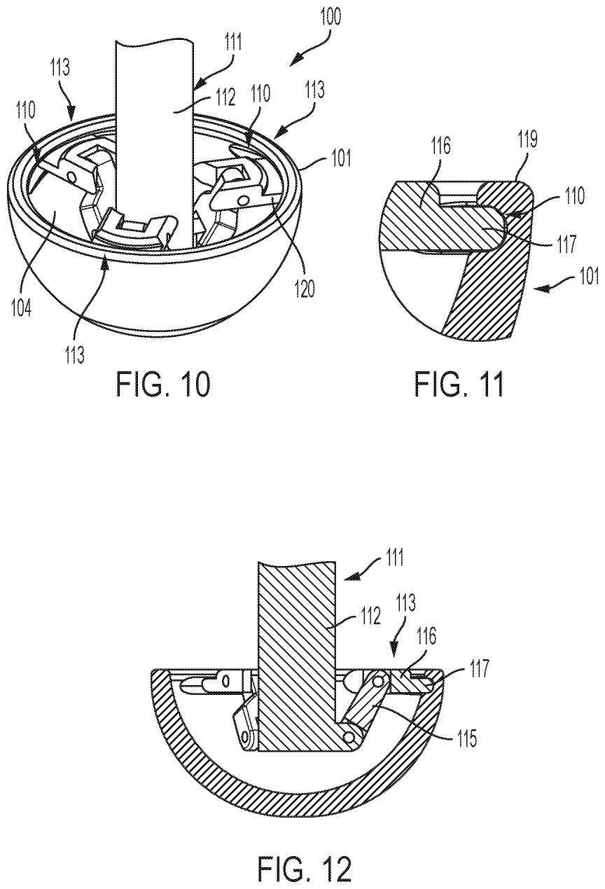

With reference to FIG. 10, an implant system 100 comprises an acetabular cup 101.

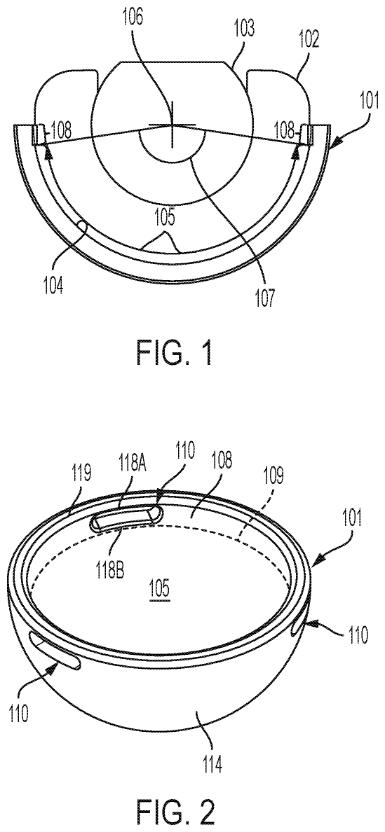

FIG. 1 shows the acetabular cup 101 engaging a dual mobility liner 102 which in turn engages a head 103 of a femoral component of a dual mobility implant. However, in alternative embodiments, the acetabular cup 101 engages the head 103 of the femoral component directly.

The acetabular cup 101 defines an interior bearing surface 104. The interior bearing surface 104 defines a central bearing zone 105 that is entirely smooth (i.e., devoid of any holes, indentations or the like). The interior bearing surface 104 may be highly polished.

The acetabular cup 101 defines a centroid of articulation 106 about which the head 103 of the femoral component rotates according to the geometry of the acetabular cup 101. Preferably, the central bearing zone 105 is within 150° of the centroid of articulation 106. In other words, with reference to FIG. 1, an interior angle 107 at the centroid 106 defined by the edge 109 of the central bearing zone 105 is preferably less than 150°.

The interior bearing surface 104 further defines a peripheral bearing zone 108 surrounding the central bearing zone 105. FIG. 2 shows an imaginary line 109 separating the central bearing zone 105 and the peripheral bearing zone 108.

The peripheral bearing zone 108 comprises a plurality of insertion tool engagement holes 110. The central bearing zone 105 does not comprise any tool engagement holes 110 and the tool engagement holes 110 are located only in the peripheral bearing zone 108.

With reference to FIG. 10, the system 100 further comprises an insertion tool 111. The insertion tool comprises a rod 112 and a plurality of radially operative interlocks 113 at a distal end of the rod 112.

The interlocks 113 are configurable in a non-engaging configuration wherein the interlocks 113 are retracted in towards the rod 112 to fit within the peripheral bearing zone 108.

FIGS. 10-12 show wherein the interlocks 113 are further configurable in an engaging configuration wherein the interlocks 113 extend out from the rod 112 to engage in respective insertion tool engagement holes 110 to attach the acetabular cup 101 to the insertion tool 111 to allow the insertion tool 111 to position the acetabular cup 111.

With reference to FIG. 12, the interlock 113 may have a distal arm 116 and a central arm 115 hingedly coupled at an angle between the distal arm 116 and the rod 112. The distal arm 116 may define an insertion portion 117 which is shaped to fit within the engagement hole 111 with little tolerance.

With reference to FIG. 5, the engagement holes 111 may be elongate and define straight upper and lower edges 118 parallel with a rim 119 of the acetabular cup 101. In this regard, the insertion portion 117 may be planar so as to fit flat against the respective edges 118 to spread insertion force along the edges 118.

The insertion portion 117 may have a rounded edge 120 (i.e., in a plane perpendicular to an elongate axis defined by the rod 112) so that the interlocks 113 can be rotated within the peripheral bearing zone 108 to find the engagement holes 110. However, the distal arm 116 preferably has sides 120 that non-releasably engage corresponding sides of the engagement hole 110 so that once the distal arm 116 has found a respective engagement hole 110, the distal arm 116 cannot be twisted out from the engagement hole 110. In this manner, the rod 112 can be twisted to rotate the acetabular cup 101. In embodiments shown in FIG. 10, the sides 120 of the distal arm 116 are planar.

As shown in FIG. 5, each tool engagement hole 101 may be approximately 12.5 mm long and approximately 3 mm wide. Furthermore, ends of the tool engagement hole 101 may have a radius of approximately 1.5 mm.

As is further shown in FIG. 4, interior edges of the engagement holes 110 may be chamfered to avoid sharp edges against the head 103 of the femoral component or the dual mobility liner 102.

The insertion tool 111 may comprise a hand operated mechanism to configure the interlocks 113 between the engaging and non-engaging configurations.

In one embodiment, the insertion tool 111 comprises a sleeve (not shown) which slides along the rod 110 to wedge between the interlocks 113. Furthermore, the interlocks 113 may be biased in towards the non-engaging configuration.

As such, when engaging the acetabular cup 111, the sleeve may be pushed to the distal end of the rod 112 which forces the interlocks 113 outward into the respective engagement holes 113 and holds the interlocks 113 in the engagement holes 113 until the sleeve is retracted.

The acetabular cup 101 is typically implanted directly in native bone of a reamed/prepared acetabulum of the hip bone. In this regard, an exterior surface 114 of the acetabular cup 101 may be coated with an osseointegration coating, such as a roughened titanium coating. Alternatively, the acetabular cup 101 may be cemented into the prepared acetabulum.

The engagement of the interlocks 113 within respective engagement holes 110 allow the insertion tool 111 to adjust the insertion angle and/or rotation of the acetabular cup 101.

Once the acetabular cup 101 is properly positioned within the acetabulum, the interlocks 113 may be retracted to the non-engaging configuration to release the engagement holes 112 to detach the insertion tool 113 from the acetabular cup 101. The insertion tool 113 is then removed from the wound, leaving the acetabular cup 101 implanted.

The insertion tool 113 may also be used during revision surgery to reposition or remove the acetabular cup 110.

The central bearing zone 105 (preferably being within 150° of the centroid of articulation 106) bears the predominant forces during articulation and is entirely smooth (i.e., devoid of holes, indentations or the like) to be able to withstand these predominant forces.

However, the peripheral bearing zone 108 only withstands minor forces or infrequent forces at extremities of rotation. The location of the engagement holes 110 only within the peripheral bearing zone 108 may only thereby have a negligible effect on the performance of the interior bearing surface 104, if at all. In other words, the location of the engagement holes 110 only within the peripheral bearing zone 108 and the central bearing zone 105 being entirely smooth allows the acetabular cup 101 to be inserted using the insertion tool 111 but without substantially degrading the performance of the bearing surface 104 (if at all) once the acetabular cup 101 is implanted.

Preferably, lower edges 118B of the tool engagement holes 110 are more than 165° apart with respect to the interior angle 107 defined by the centroid 106.

In embodiments, the upper edges 118A of the tool engagement holes 118 define an interior angle of less than 180° to allow for a non-constrained-type acetabular cup 101.

In a preferred embodiment, the acetabular cup 113 is ceramic. As alluded to above, an exterior surface 114 of the acetabular cup 111 may be coated with an osseointegration coating.

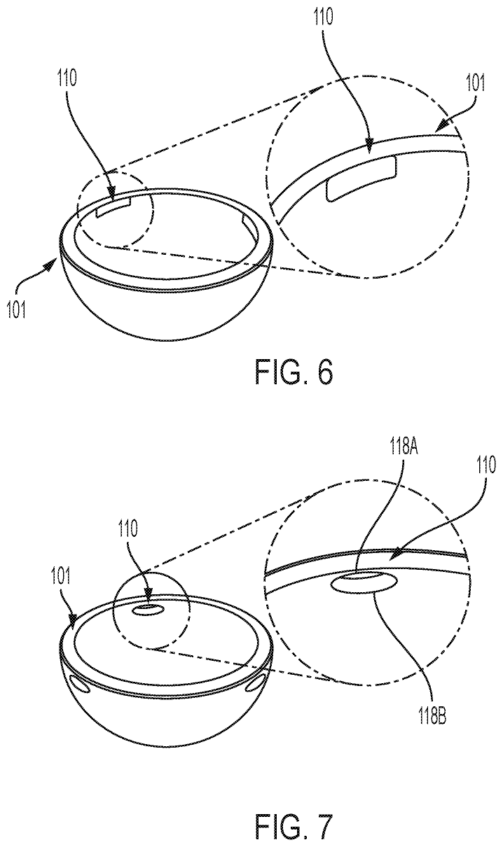

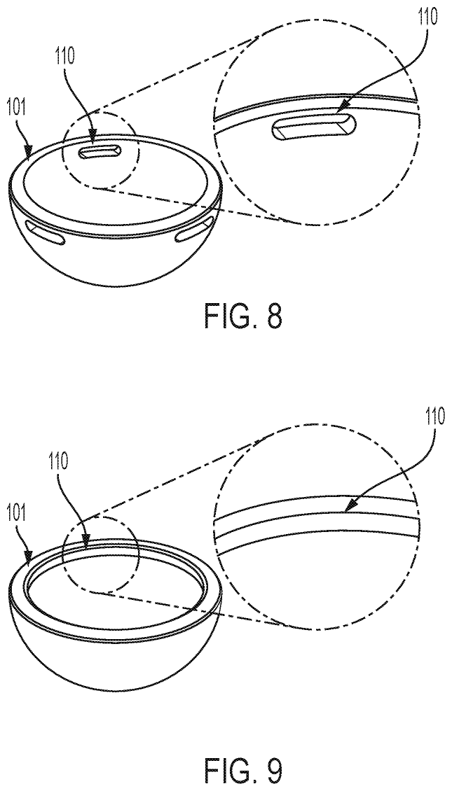

FIG. 8 shows a preferred embodiment of the acetabular cup 101 wherein the engagement holes 110 are through holes. Finite element analysis suggests that through holes may reduce structural stresses of a ceramic acetabular cup 101, especially during sintering. However, FIG. 6 shows an alternative embodiment wherein the engagement hole 110 is a blind hole. In other words, the blind hole 110 is an indentation in the interior bearing surface 104, but does not transect the exterior surface 114.

FIGS. 6 and 8 show wherein the engagement holes 110 have straight upper and lower edges 118. However, the embodiment of FIG. 7 shows an embodiment wherein the engagement hole 110 is ovular in cross-section wherein the upper and lower edges 118 thereof have curvature.

FIG. 9 shows an embodiment of the acetabular cup 101 wherein the acetabular cup 101 comprises a single engagement hole 110 in the form of a channel around the entire periphery of the peripheral bearing zone 108. However, this embodiment is less preferred as it would be difficult to rotate the acetabular cup 101.

In embodiments, the insertion tool may comprise an impact plate (not shown) which is located above the interlocks 113 and which is configured to lie flat against the rim 119 of the acetabular cup 101. Furthermore, the offset of the interlocks 113 from the impact plate may be configured so that when the impact plate lies against the rim 119, the interlocks 113 are appropriately offset to find the engagement holes 110.

The impact plate may be used spread impact force around the rim 119 of the acetabular cup 101. As such, the rod 112 may be slapped or struck to drive the acetabular cup 101 into the acetabulum by the impact plate without loading the interlocks 113.

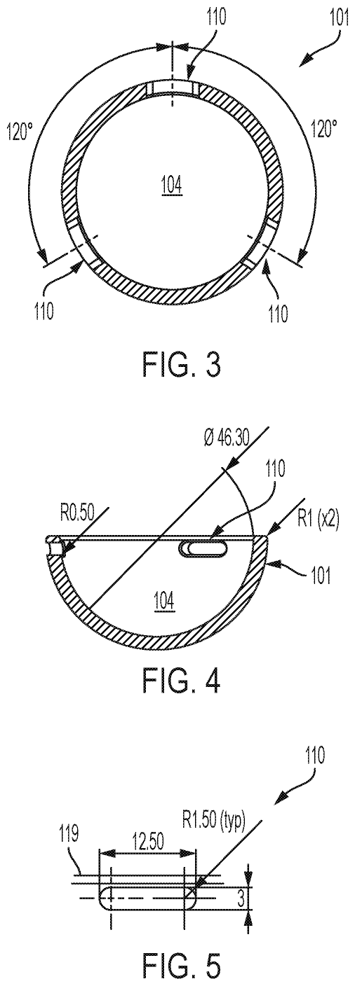

In a preferred embodiment as is shown in FIG. 3, the acetabular cup 101 comprises three engagement holes 108. Three engagement holes 110 represents the minimum amount of holes 110 (i.e., minimising the effect on the interior bearing surface 104) which yet allows the insertion tool 101 to manipulate the acetabular cup 101 along three axes.

As is further shown in FIG. 3, the engagement holes 100 may be equidistantly spaced (i.e., spaced apart by 120°)

The foregoing description, for purposes of explanation, used specific nomenclature to provide a thorough understanding of the invention. However, it will be apparent to one skilled in the art that specific details are not required in order to practise the invention. Thus, the foregoing descriptions of specific embodiments of the invention are presented for purposes of illustration and description. They are not intended to be exhaustive or to limit the invention to the precise forms disclosed as obviously many modifications and variations are possible in view of the above teachings. The embodiments were chosen and described in order to best explain the principles of the invention and its practical applications, thereby enabling others skilled in the art to best utilize the invention and various embodiments with various modifications as are suited to the particular use contemplated. It is intended that the following claims and their equivalents define the scope of the invention.

1. An implant system comprising:
an acetabular cup defining:
an interior bearing surface, the interior bearing surface defining:
a central bearing zone that is entirely smooth;
a peripheral bearing zone surrounding the central bearing zone; and
a plurality of insertion tool engagement holes located only in the peripheral bearing zone; and
an insertion tool comprising:
a rod;
a plurality of radially operative interlocks at a distal end of the rod, the interlocks configurable in:
a non-engaging configuration wherein the interlocks are retracted in towards the rod to fit within the peripheral bearing zone; and
an engaging configuration wherein the interlocks extend out from the rod to engage respective insertion tool engagement holes to attach the acetabular cup to the insertion tool for the insertion tool to position the acetabular cup.

2. The system as claimed in claim 1, wherein the central bearing zone is entirely within 150° of a centroid of articulation of the interior bearing surface.

3. The system as claimed in claim 1, wherein the lower edges of the tool engagement holes are more than 165° apart with respect to an interior angle defined by the centroid.

4. The system as claimed in claim 1, wherein upper edges of the tool engagement holes are less than 180° apart with respect to an interior angle defined by the centroid.

5. The system as claimed in claim 1, wherein the peripheral bearing zone comprises three tool engagement holes.

6. The system as claimed in claim 5, wherein the tool engagement holes are equidistantly spaced.

7. The system as claimed in claim 1, wherein the acetabular cup is ceramic.

8. The system as claimed in claim 1, wherein the acetabular cup has a roughened exterior osseointegration surface.

9. The system as claimed in claim 1, wherein each interlock has a distal arm and a central arm hingedly coupled at an angle between the distal arm and the rod.

10. The system as claimed in claim 1, wherein each interlock defines an insertion portion which is shaped to fit within the engagement hole with little tolerance.

11. The system as claimed in claim 10, wherein the engagement holes are elongate and define straight upper and lower edges parallel with a rim of the acetabular cup and wherein the insertion portion is planar.

12. The system as claimed in claim 10, wherein the insertion portion has a rounded edge in a plane perpendicular to an elongate axis defined by the rod.

13. The system as claimed in claim 10, wherein the insertion portion has sides that non-releasably engage corresponding sides of an engagement hole when the insertion tool is twisted with respect to the acetabular cup.

14. The system as claimed in claim 1, wherein each engagement hole is more than 10 mm long.

15. The system as claimed in claim 1, wherein each engagement hole is less than 5 mm wide.

16. The system as claimed in claim 1, wherein interior edges of each engagement holes is chamfered.

17. The system as claimed in claim 1, wherein the insertion tool comprises a hand operated mechanism to configure the interlocks between the engaging and non-engaging configurations.

18. The system as claimed in claim 17, wherein the insertion tool comprises a sleeve which slides along the rod to wedge between the interlocks to forces the interlocks outward into respective engagement holes and hold the interlocks in the respective engagement holes.

19. The system as claimed in claim 18, wherein the interlocks are biased to the non-engaging configuration.

20. The system as claimed in claim 1, wherein the insertion tool engagement holes are through holes.

21. The system as claimed in claim 1, wherein the insertion tool comprises an impact plate which is configured to lie flat against the rim of the acetabular cup.

22. The system as claimed in claim 21, wherein an offset of the interlocks from the impact plate is configured so that when the impact plate lies against the rim, the interlocks are appropriately offset to find the engagement holes.

23. A method using the system as claimed in claim 1, the method comprising:
configuring the interlocks in the non-engaging configuration;
inserting the interlocks within the peripheral bearing zone;
configuring the interlocks in the engaging configuration so that the interlocks extend out from the rod to engage respective insertion tool engagement holes to attach the acetabular cup to the insertion tool; and
using the insertion tool to position the acetabular cup.

24. An acetabular cup defining an interior bearing surface, the interior bearing surface defining:
a central bearing zone that is entirely smooth;
a peripheral bearing zone surrounding the central bearing zone; and
a plurality of insertion tool engagement holes located only in the peripheral bearing zone.
